(19) United States
(12) Patent Application Publication
(10) Pub. No.: US 2013/0302387 A1
(43) Pub. Date: Nov. 14, 2013

(54) Compositions and Formulations for the Treatment of Halitosis

(71) Applicant: EJP Pharmaceutical ApS, (US)

(72) Inventor: Ejvind Jersie PEDERSEN, Skodsborg (DK)

(21) Appl. No.: 13/893,588

(22) Filed: May 14, 2013

Related U.S. Application Data
(60) Provisional application No. 61/646,508, filed on May 14, 2012.

(51) Int. Cl. A61K 8/58; A61Q 11/00

(52) U.S. Cl. CPC A61K 8/58; A61Q 11/00
USPC 424/401; 556/134; 424/49; 424/48

(57) ABSTRACT

An orally disintegrating composition comprising a chelate comprising a metal ion and a biologically acceptable amino acid and having the general formula

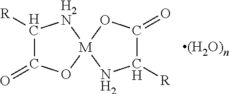

wherein M is the metal ion Zn²⁺ and R is H in the biologically acceptable amino acid glycine, use of the composition for the treatment of halitosis, and a chewing gum for the treatment of halitosis comprising a chelate comprising a metal ion and a biologically acceptable amino acid and having said general formula, which chewing gum is based on a carrier composition comprising a gum base, sorbitol, xylitol, one or more plasticizer(s), and one or more anticaking agent(s).

### FIELD OF INVENTION

The present invention relates to mouth hygienic compositions and formulations, which are suitable for the treatment of halitosis. The present invention relates in particular to orally disintegrating compositions and chewing gums in which amino chelated zinc suitable for the treatment of halitosis, e.g. a zinc bisglycinate chelate, is incorporated.

### BACKGROUND OF THE INVENTION

It is widely accepted that for many people the affliction of halitosis (bad breath) may constitute a serious problem, particularly in social encounters. The breath malodour may be very severe and it may occur e.g. occasionally, regularly, or chronically and at specific times of the day or month. For the purposes of this application, the term “halitosis” means an unpleasant breath odour that is objectionable to others.

Studies on the etiologies of breath malodour agree that hydrogen sulphide (H₂S), methyl mercaptan (CH₃SH), and dimethyl sulphide (CH₃SCH₃), collectively referred to as volatile sulphur compounds (VSCs) are the principal odourants in halitosis. VSCs originate from the anaerobic bacterial degradation of sulphur-containing amino acids within the oral cavity. It is now generally accepted that VSCs constitute the major component of halitosis originating from the oral cavity. It has also been shown that anaerobic, Gram negative bacteria are responsible for this odour production.

Consequently, all conditions which favour the retention of such a microbial flora predispose for the formation of VSC and thereby contribute to the development of halitosis. As substrates for odour production, the bacteria mainly utilize the amino acids methionine and cysteine present in e.g. proteins from a dietary intake. These amino acids contain sulphur and are metabolized by the bacteria to yield VSCs. These substances have an unpleasant odour, even in extremely low concentrations.

### DESCRIPTION OF THE PRIOR ART

WO2010104563A2 relates to a highly compactable and durable solid dispersion, and excipient system made therefrom, comprising co-processed carbohydrates which have different solubilities and/or concentrations, and microcrystalline plate structure, and formulations produced therefrom, which formulations are directly compressible into solid dosage forms. WO2010104563A2 also relates to orally disintegrating tablets comprising several different polyols with different solubilities, lubricant(s), disintegrant(s) and an active agent, which could be coated or spray dried. WO2010104563A2 does neither disclose nor suggest that amino chelated zinc suitable for the inhibition of halitosis, e.g. zinc bisglycinate chelate, could be incorporated into said tablets.

WO06058250A2 discloses orally disintegrating compositions as well as active ingredients in a solid pharmaceutical composition comprising several polyols, i.e. sugar alcohols. The sole exemplified active ingredient is rasagiline, an amine commonly used in the treatment of e.g. Parkinson's disease. WO2010104563A2 does neither disclose nor suggest that amino chelated zinc suitable for the inhibition of halitosis, e.g. zinc bisglycinate chelate, could be incorporated into said tablets.

WO9917735A1 relates to mouth hygienic compositions for the treatment of e.g. halitosis. It also relates to the use of metal chelates in said composition and a method for using the composition. WO9917735A1 discloses a number of different formulations suitable for releasing the chelate into the oral cavity. WO9917735A1 also discloses that inter alia lozenges, toothpaste and liquid mouth-rinsing composition are suitable formulations. Chewing gums are also mentioned but not exemplified in an enabling manner. WO9917735A1, however, does neither disclose nor suggest that amino chelated zinc suitable for the inhibition of halitosis, e.g. zinc bisglycinate chelate, could be incorporated in an orally disintegrating composition or a chewing gum according to the present invention.

Additionally, it is known that aqueous solutions of zinc salts used as mouth rinses reduce and inhibit VSC formation in the oral cavity. It is assumed that zinc ions form stable mercaptides with the substrate, with precursors of VSC or with the VSC directly, since zinc has an affinity for sulphur and oxidizes sulphhydryl groups. It has for example been established that zinc-containing chewing gum has an effect on VSCs in the oral cavity (S. M. Wäler: The effect of zinc-containing chewing gum on volatile sulphur-containing compounds in the oral cavity; Acta Odontol. Scand. 55 (1997)).

In a halimetric test study carried out on 80 subject all suffering from halitosis, it was concluded, based on the results from a halimeter device, i.e. an apparatus which determines the VSC concentration in the expired air, that up till 6 hours following treatment (sucking) of a zinc bisglycinate based tablet (sold under the trade name Hali-Z) a significant reduction of the degree of halitosis was observed in the subjects. Moreover, the test report also discloses that a significant reduction in the levels of VSC was observed up till 6 hour after treatment with said tablet which, in turn, evidences the direct relationship between VSC and halitosis (Study report by Specialist Dental Care Centre, Pomeranian Medical University in Szczecin).

Several examples of compounds suggested to be effective as halitosis inhibitors are described in the prior art. As an example, Canadian patent application no. 2,154,860 relates to an oral care product which contains alkali metal pyrophosphate and a water-soluble zinc polyamine complex capable of releasing zinc ions in an environment such as the oral cavity. The zinc polyamine complex is formed from a polyamine and a normally water-insoluble zinc compound such as zinc oxide or zinc citrate. The aim is to provide a high-molecular weight water-soluble polyamine complex of a normally water-insoluble zinc compound which has utility as an ingredient of improved palatability and reduced astringency in oral care products.

The water-soluble zinc polyamine complex is present in an aqueous solution which has a clear transparency and is without any visible evidence of a second phase which is distinct from the aqueous phase. Reference is made to the fact that the polyamines cited in the above-mentioned Canadian patent application have an average molecular weight of about 1,500 to 70,000. The invention described in Canadian patent application no. 2,154,860 is significantly different from the present invention, both in terms of the solubility of the zinc compound and in terms of the molecular weight of the composition used.

European patent application no. 0 522 965 A1 discloses a composition for use in the treatment of e.g. halitosis. The composition does not comprise a chelate of an amino acid with a metal ion.

U.S. Pat. No. 4,814,163 relates to a solid antitartar and mouth deodorant composition comprising a physiologically acceptable zinc compound, an ionone ketone terpene derivative, a mint flavour and a sodium or potassium gluconate, and having an acidic pH, in a sugar-free carrier. U.S. Pat. No. 4,814,163 does not disclose a mouth hygienic composition comprising a chelate of a metal ion with an amino acid.

In general, when metals such as zinc, manganese, magnesium, copper, iron, cobalt and others become surrounded by and bonded to amino acids, in a stable form, this is referred to as chelation or chelate formation. Such chelates are referred to in the art as e.g. metal amino acid chelates, mineral amino acid chelates and chelates comprising a metal ion and one or more amino acids. Furthermore, chelates are also often referred to in the art as so-called coordination compounds. The coordination compounds are very often slightly soluble, non-ionic complexes.

Chelation is the natural means for the body to transport minerals across the intestinal wall as part of digestion. The body is very efficient at absorbing amino acids in this way. In a priority list of nutritional substances crossing the intestinal wall after digestion, amino acids rank highly. In fact, 95% of all amino acids are absorbed. Chelating minerals such as metal ions to these amino acids facilitates the transport of the mineral across the intestinal wall. In this respect it is very important for the mineral to have a stable bond to the amino acid.

U.S. Pat. No. 5,516,925 relates to mineral amino acid chelates specifically as supplementary mineral sources for use in human or animal nutrition. It does not relate to a mouth hygienic composition, but is concerned with facilitating the absorption in the gut and mucosal cells of the amino acid chelate.

Water-soluble as well as water-insoluble zinc compounds have also been utilized as physiologically active ingredients in oral care preparations.

Water-soluble and highly ionized zinc compounds, such as zinc chloride, would appear to provide a valuable source of bioavailable zinc ions. However, zinc chloride in aqueous solution tends to form oxychloride and zinc hydroxides of low solubility, which results in a two phase, cloudy solution.

The pH of a conventional zinc chloride solution can be lowered to less than 4.5 through the use of mineral or organic acid buffers to provide a stable and clear solution. However, this method is not acceptable since the resultant oral care product exhibits severe astringency and an undesirable sour taste.

Other zinc salts, such as e.g. zinc acetate and zinc citrate, have been used for the prevention of halitosis. However, zinc acetate and zinc citrate also have a high degree of astringency and an undesirable metallic taste.

As a consequence of these undesirable characteristics it would be desirable to provide the zinc-containing compound as part of a mouth hygienic composition which is controllably releasable into the oral cavity of a subject so as to provide an effective contact between the zinc and the VSCs present in this environment. Thus, in light of the above the present invention for the first time provides the possibility to incorporate an amino zinc bisglycinate chelate into orally disintegrating compositions and chewing gums in order to enable the user to combat halitosis by a number of alternative and user-friendly formulations, i.e. as alternatives to e.g. traditional mouth-washes, toothpastes etc.

### SUMMARY OF THE INVENTION

The present invention relates to amino chelated zinc, e.g. zinc bisglycinate chelate, incorporated into an orally disintegrating composition or a chewing gum having the characteristics of the present invention.

As regards the orally disintegrating composition, the inventor of the present invention has surprisingly found that amino chelated zinc, e.g. zinc bisglycinate chelate, can be effectively and organoleptically acceptably incorporated into orally disintegrating compositions provided that the carrier of said composition e.g. comprises between 70 and 85 wt % of polyols having average particle sizes between 90-120 μm. This may be achieved by making use of commercially available polyol compositions such as ADVANTOL™ 300 (from SPI Pharma, USA) and/or PEARLITOL® Flash (from Roquette, France).

As regards the incorporation of the amino chelated zinc, e.g. zinc bisglycinate chelate, into the chewing gum of the present invention, it has surprisingly been found that obtaining an organoleptically acceptable chewing gum based on amino chelated zinc, e.g. zinc bisglycinate chelate, requires a particular carrier composition comprising specific amounts of gum base, sorbitol, xylitol, plasticizers and anticaking agents, such as magnesium stearate and/or talc powder or similar glidants or antiadherants known to the skilled person in the art.

### DETAILED DESCRIPTION OF THE INVENTION

### The Orally Disintegrating Composition

Orally disintegrating compositions, e.g. in form of orally disintegrating tablets (ODTs) are designed to disintegrate rapidly on contact with saliva in the oral cavity and enable inter alia oral treatment without use of water or chewing. Additionally, these formulations offer increased convenience and ease of administration with the potential to improve compliance, particularly in certain populations where swallowing conventional solid oral-dosage forms presents difficulties.

The orally disintegrating compositions of the present invention also covers ODTs, e.g. in form of a cosmetic composition, wherein the active ingredient, i.e. the amino chelated zinc, is spat out after completions of the mouth wash. In effect, such an ODT will in principle be a mouth rinse product without water, i.e. a water-free mouth rinse ODT. Spitting out the amino chelated zinc after mouth washing is desirable if e.g. no dietary supplementation of zinc is needed/wanted or if the zinc amount of the ODT is exceeding the acceptable daily single and/or total zinc doses.

Further, ODTs of the present invention can be used where it is inconvenient to bring and/or use traditional mouth rinses known in the art.

Even further, ODTs of the present invention can be used either alone, i.e. dissolving in oral cavity solely in the saliva or by adding a little water.

An ODT has traditionally been defined as a solid-dosage form containing medicinal substances which disintegrates rapidly, usually within a matter of seconds, when placed on the tongue. Over the past few years, ODT technologies have increasingly been used within the pharmaceutical sector as an alternative dosage form for patients who experience dysphagia (difficulty in swallowing) or for patients where compliance is a known issue and therefore the easier ODT dosage form ensures that medication is taken.

According to the state of the art, the paramount focus of ODTs has thus been to facilitate systemic drug administration to dysphagia patients, by applying orally disintegrating compositions, preferably ODTs in the form of tablets or similar formulations.

Applying orally disintegrating compositions, e.g. in the form of ODTs for mouth-hygienic purposes, in e.g. treatment of halitosis, where the mode of action is local within the oral cavity, has not previously been disclosed. In effect, the orally disintegrating compositions and/or formulations of the present invention would constitute a suitable alternative to or replacement of conventional mouthwash or mouth rinse products.

### The Carrier Used in the Orally Dissolving Composition and Formulation

It has surprisingly been found that the composition and the ratios between individual constituents of the carrier are essential for providing an organoleptically acceptable formulation suitable for delivering aminochelated zinc, e.g. zinc bisglycinate, into the oral cavity.

It has been found that the carrier preferably shall comprise between 70 and 85 wt % of polyols having average particle sizes between 90-120 μm. Additionally, it has been found that polyols preferably shall be chosen from sorbitol, mannitol, xylitol, erythritol, maltitol, lactitol, isomalt and/or mixtures thereof. Even further it has been found that organoleptically acceptable formulations is obtainable if the amounts of the individual polyol constituents are: 70-99.5 wt % mannitol, 0.5-30 wt % sorbitol, 0-5 wt % xylitol, 0-5 wt % erythrol, 0-30 wt % maltitol, 0-5 wt % lactitol, 0.5-20 wt % isomalt of the total amount of polyols.

A preferred carrier for use in the ODT formulation according to the invention is a commercially available carrier with the trade name ADVANTOL™ 300 (from SPI Pharma, USA).

Another preferred carrier for use in the formulation according to the invention is a commercially available carrier with the trademark PEARLITOL® Flash (from Roquette, France), which comprises approximately 80% mannitol (CAS no. 69-65-8) and approximately 20% maize starch (CAS no. 9005-25-8)

The present formulation may also comprise, alternativly or additionally, a carbohydrate system of the kind described in WO 03/051338.

### The Carrier Used in the Chewing Gum

It has surprisingly been found that the composition and the ratios between individual constituents of the carrier are essential for providing an organoleptically acceptable chewing gum formulation suitable for delivering aminochelated zinc, e.g. zinc bisglycinate, into the oral cavity. To obtain such a formulation it was found that the carrier composition preferably should comprise 28-31 wt % of a gum base, up to 100 wt % of sorbitol, 8-12 wt % xylitol, ≦1.5 wt % of one or more plasticizer(s) and ≦2.0 wt % of one or more anticaking agent(s).

Additionally it was found that obtaining an organoleptically acceptable formulation required that all constituents of the carrier composition should have particle sizes allowing passage through a sieve size of at least 500 μm.

A preferred carrier for use in the chewing gum formulation according to the invention is a commercially available carrier with the trade name, Cafosa HiG PWD-01.

An even more preferred carrier for use in the chewing gum formulation according to the invention is a commercially available carrier with the trade name, Cafosa HiG PWD-02.

### The Polyols

Polyols, or sugar alcohols, a class of polyols, are commonly added to foods because of their lower caloric content than sugars. They are also added to chewing gum because they are not broken down by bacteria in the mouth or metabolized to acids, and thus do not contribute to tooth decay. Maltitol, sorbitol, xylitol and isomalt are some of the more common types of polyols.

Suitable polyols according to the present invention may include but are not limited to sugar alcohols of the general formula CH₂OH-(CHOH)n-CH₂OH, where n is 2 to 6, and preferably 3 to 6, and their dimeric anhydrides. In some embodiments, the polyols include, but are not limited to sorbitol, mannitol, xylitol, erythritol, maltitol, lactitol, isomalt, and mixtures thereof.

### The Chelate

The chelate of the present invention is amino chelated zinc, e.g. zinc bisglycinate chelate, of the general formula:

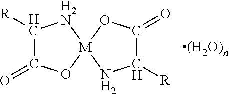

wherein M is the metal ion Zn²⁺ and R represents H in the biologically acceptable amino acid glycine.

Zinc bisglycinate has CAS No. 14281-83-5.

It is desirable that the reaction leading to the chelate formation takes place under conditions characterized by e.g. a molar ratio such as one mole of zinc ion to one to three, preferably two, moles of glycine. The resulting chelate differs from traditional salts by having different physical and chemical properties such as e.g. the nature of the chemical bonds involved in forming the different chemical structures.

It should be noted that a chelate is not the same as a complex or, indeed, a complex mixture of a mineral and a protein hydrolysate.

Consequently, simply mixing inorganic minerals with amino acids in a liquid or dry mixture does not fall into the category of a true amino acid chelate. Such a simple ionic and hydrogen bonding of minerals to amino acids does not produce a stable product. Special processing must be performed to create a stable (covalent) bond, which is important for greater bioavailability.

When forming a product with e.g. a mineral, such as a metal ion, the complex is termed a chelate. Even if the amino chelated zinc, e.g. zinc bisglycinate, is relatively easily soluble in water, the zinc-component is strongly bound to the amino acid, e.g. the glycerine, of the chelate. This characteristic can be exploited e.g. when providing a composition wherein the chelate is to be controllably releasable into the oral cavity of a subject.

The chelate according to the present invention is particularly useful in treating halitosis while at the same time having pleasant organoleptic qualities and being essentially tastefree in the absence of a flavouring agent.

The high stability constant of the chelate of the present invention can be exploited e.g. when providing a mouth hygienic composition which is to be slowly dissolved in an aqueous environment, such as in the saliva of the oral cavity. It is important that the active ingredient of the composition, the amino chelated zinc, e.g. zinc bisglycinate, may suitably be released under controllable conditions which facilitate an effective interaction of the zinc ion with the halitosis causing volatile sulphur compounds present in an oral cavity environment. This effective interaction desirably takes place without the generation of any astringent taste or unpleasant smell. Preferably, the composition according to the present invention is substantially tasteless unless deliberately being supplemented with a desirable flavouring agent.

Chelates which are useful in the present invention are commercially available and can be prepared by following the techniques generally available in the art of chelate preparation. As an example, reference can be made to the method of preparing amino acid chelates disclosed by Ashmead in U.S. Pat. No. 4,830,716.

Zinc is a particularly useful metal in the context of the present invention, as the zinc ion, Zn²⁺, of the chelate is releasable under controllable conditions in the oral cavity and thus readily available for reacting with VSCs.

Even if in principle any biologically acceptable amino acid can be used in the preparation of metal amino acid chelates according to the present invention, glycine is the preferred one. A glycine-Zn²⁺ chelate has shown to be exceptionally effective in treating halitosis.

In one embodiment of the invention, Zn²⁺ is preferably present in the mouth hygienic composition, i.e. the orally disintegrating composition as well as the chewing gum, in an amount of 0.05 to 4.0 wt %, such as 0.1 to 3.9 wt %, more preferably 0.2 to 3.8 wt %, such as 0.4 to 3.7 wt %, even more preferably 0.6 to 3.6 wt %, such as 0.8 to 3.4 wt %, and most preferably 1.0 to 3.3 wt %, such as 2 wt %.

### The Orally Disintegrating Formulations

The formulations of the present invention may be tableted in conventional tabletting devices, e.g. compressing into tablets by conventional rotary tablet compressing machines. The tabletting tool (punches and dies) should be lubricated by means of suitable lubricant, such as magnesium stearate.

The amino chelated zinc composition and formulation of the present invention may be flavoured with a flavouring agent to make it more palatable. Suitable flavouring agents are those generating a flavour of e.g. lemon, strawberry, raspberry, peach, blackcurrant, orange, cherry, peppermint or menthol. Raspberry flavouring agents are particularly preferred due to their ability to provide particularly pleasing organoleptic qualities and their ability to reduce and/or eliminate any traces of an astringent taste associated with the zinc amino acid chelate.

Apart from being palatable, it is also desirable that the composition is capable of releasing the chelate in an aqueous environment, such as e.g. the oral cavity, under controllable conditions, such as e.g. slowly and/or at a steady rate. To facilitate the formation of such an environment the composition may comprise a saliva-inducing agent such as e.g. sorbitol and/or xylitol in a suitable ratio in order to stimulate the production of saliva in the oral cavity. This stimulation will facilitate the slow and/or controlled release of the chelate from the composition.

Such a preparation or formulation is suitably an orally disintegrating composition in the form of a tablet, a lozenge or a troche. Such formulations can be prepared by directly applying the amino chelated zinc, e.g. zinc bisglycinate, to the specific carrier composition of the present invention, e.g. commercially available carrier, Advantol™ 300, followed by conventional dry blending and compressing techniques, e.g. in a standard rotary tablet press with standard tooling under normal tabletting temperature and humidity conditions, which is well-known in the art.

See under EXAMPLES for more details.

### The Chewing Gum Formulations

A chewing gum formulation according to the present invention can be prepared by conventional tabletting devices, e.g. conventional rotary tabletting apparatus. The tabletting tool should be lubricated by means of suitable lubricant, such as magnesium stearate.

See under EXAMPLES for more details.

### Advantages of Chewing Gum Comprising Amino Chelated Zinc

Compared to prior art dentifrices (i.e. mouth hygienic paste, liquids or powders), mouth rinses and toothpaste, which could reasonably be categorized as mouth hygienic compositions with relatively short exposure times in the oral cavity, chewing gum could be categorized as mouth hygienic compositions with relatively long exposure times in the oral cavity.

The chewing gum may also be used for treatment against xerostomia.

Additionally, compared to e.g. toothpaste and mouth rinses, treatment of halitosis with amino chelated zinc based chewing gum would provide for a larger exposure area in the oral cavity, i.e. including also major parts of the throat (i.e. the pharynx and the tonsils).

Thus, while prior art dentifrices (i.e. mouth hygienic paste, liquids or powders), mouth rinses and toothpaste are merely effecting the anterior part of the oral cavity chewing gum would—after dissolution of the therapeutically active zinc chelate in the saliva and after subsequent swallowing—provide for a halitosis-reducing effects also in the posterior parts of the oral cavity, i.e. including major areas of the pharynx and the tonsils as well, i.e. the parts of the oral cavity mainly responsible for the formation of VSCs and thus halitosis (Rosenberg M., JADA, vol 127, April 1996, p. 476).

As an additional effect, zinc amino acid chelates such as e.g. zinc bisglycinate chelate do not possess the undesirable metal-like taste and high degree of astringency which are typical of the zinc salts used in numerous prior art formulations. Therefore the present invention also provides an organoleptically acceptable mouth hygienic composition which is effective in the treatment of halitosis.

### Advantages of Orally Disintegrating Compositions Comprising Amino Chelated Zinc

The advantages outlined for chewing gum comprising amino chelated zinc apply as well for orally disintegrating compositions comprising amino chelated zinc.

An additional advantage of using orally disintegrating compositions or formulations comprising amino chelated zinc, e.g. in form of form of a lozenge, a tablet, a pellet, a pill, troche or a water-free mouth rinse ODT, is inter alia improved user-friendliness compared to e.g. traditional mouth-washes.

### Example 1

The following examples illustrate the invention. Although the components and the specific ingredients are presented as being typical, various modifications within the scope of the invention can be derived based on what is disclosed in the above description.

A first embodiment of the present invention relates to an orally disintegrating composition comprising a chelate comprising a metal ion and a biologically acceptable amino acid and having the general formula

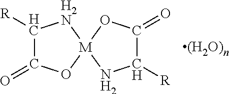

wherein M is the metal ion Zn²⁺ and the biologically acceptable amino acid is glycine.

In preferred embodiments of the resent invention the orally disintegrating composition has the following additional characteristics, either alone or in combination:
an orally disintegrating composition wherein said chelate is controllably releasable into the oral cavity of a subject;
an orally disintegrating composition, wherein the composition is based on one or more polyols, optionally in combination with starch, such as maize starch;
an orally disintegrating composition wherein the composition is based on one or more polyols having average particle sizes of 90-120 μm;
an orally disintegrating composition wherein the total content of polyols in the composition is between 70 and 85 wt %;
an orally disintegrating composition wherein the polyols are sorbitol, mannitol, xylitol, erythritol, maltitol, lactitol, isomalt and/or mixtures thereof;
an orally disintegrating composition wherein the individual amounts of polyols are: 70-99.5 wt % mannitol, 0.5-30 wt % sorbitol, 0-5 wt % xylitol, 0-5 wt % erythrol, 0-30 wt % maltitol, 0-5 wt % lactitol, 0.5-20 wt % isomalt of the total amount of polyols. Said orally disintegrating composition can comprise all possible combinations of any of said individual amounts;
an orally disintegrating composition comprising Zn²⁺ in an amount of 0.05 to 4.0 wt %, such as 0.1 to 3.9 wt %, more preferably 0.2 to 3.8 wt %, such as 0.4 to 3.7 wt %, even more preferably 0.6 to 3.6 wt %, such as 0.8 to 3.4 wt %, and most preferably 1.0 to 3.3 wt %, such as 2 wt %;
an orally disintegrating composition which is completely dissolved in the oral cavity within 180 seconds upon exposure to the saliva, preferably within 60 seconds upon exposure to the saliva;
an orally disintegrating composition comprising one or more saliva-inducing agent(s);
an orally disintegrating composition comprising one or more flavouring agent(s);
an orally disintegrating composition wherein the flavouring agent(s) includes a mouth refreshing agent such as levomentholum, coolmint flavours or similar menthol based compounds made from peppermint or other mint oils;
an orally disintegrating composition in the form of a lozenge, a tablet, a pellet, a pill or a troche.

In an additional embodiment the present invention relates to the use of an orally disintegrating composition with any of the above-outlined characteristics for the treatment of halitosis.

### Example 2

6.8 g of zinc bisglycinate (10%), batch no. 1059888, i.e. corresponding to 6.8 mg zinc per tablet, was added directly into 49.04 g the commercially available carrier Advantol™ 300 (lot 121101344, prod. code 112-1234). No pre-treatment in the form of e.g. pulverizing, sieving or the like was required, neither for the chelate nor the carrier.

The mixture of zinc bisglycinate and Advantol™ 300 was dry blended and subsequently transferred to a manually operable tabletting apparatus (Diaf Type TWA4 Special) wherein the ODTs were compressed under pressure, i.e. approx. 1 metric ton. Tabletting was carried out under normal tabletting temperature, i.e. approx. 20-22° C., and humidity conditions, i.e. approx. 60% relative humidity (RF).

Manufacturing at temperatures of approx. 18-22° C. (room temperature) and a humidity of approx. 20% RF has turned out to provide an improved maintenance of the hardness of the ODT during manufacture and might be used for industrial scale manufacturing.

The mixture resulted in 100 ODTs having a weight of approx. 500 mg/ODT. The tablet hardness obtained was approx. 8-10 Kp. The resulting ODT was capable of completely dissolving in water (37° C.) as well as in the saliva of the oral cavity in less than approx. 60-75 seconds.

The resulting ODTs had excellent organoleptic properties.

### Example 3

In a further embodiment of the present invention, tabletting was carried out under the same conditions as in EXAMPLE 2, albeit a rotary tablet press having the trade name FETTE™ 1200 was applied. The tabletting tool was lubricated by means of spraying magnesium stearate onto the punches and dies.

The resulting ODTs had excellent organoleptic properties.

### Example 4

A preferred chewing gum according to the present invention is a chewing gum for the treatment of halitosis comprising a chelate comprising a metal ion and a biologically acceptable amino acid and having the general formula

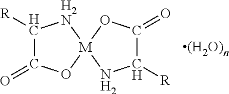

wherein M is the metal ion Zn²⁺ and the biologically acceptable amino acid R is glycine.

The chewing gum is based on a carrier composition comprising:

(i) a gum base, (ii) sorbitol, (iii) xylitol, (iv) one or more plasticizer(s) and (v) one or more anticaking agent(s).

In particular, in order to be able to suitably incorporate the amino chelated zinc into an organoleptically acceptable chewing gum, the carrier composition should be as follows:

(i) a gum base: 28-31 wt %

(ii) sorbitol: up to 100 wt %

(iii) xylitol: 8-12 wt %

(iv) one or more plasticizer(s): ≦1.5 wt %

(v) one or more anticaking agent(s): ≦2.0 wt %
based on the total weight of the carrier composition.

In preferred embodiments of the present invention the chewing gum has the following additional characteristics, either alone or in combination:
a chewing gum wherein the chelate is controllably releasable into the oral cavity of a subject,
a chewing gum comprising one or more saliva-inducing agent(s);
a chewing gum comprising one or more flavouring agent(s);
a chewing gum wherein the flavouring agent(s) includes an artificial sweetener such as acesulfam potassium;
a chewing gum wherein the flavouring agent(s) includes a mouth refreshing agent such as levomentholum, coolmint flavours or similar menthol based compounds made from peppermint or other mint oils;
a chewing gum comprising one or more flow agent(s) such as colloidal silica;
a chewing gum according wherein the ratio between the chelate, the carrier composition and any excipients are approximately 2:94:4;
a chewing gum wherein all exipients have particle sizes allowing passage through a sieve size of at least 710 μm;
a chewing gum wherein all constituents of the carrier composition have particle sizes allowing passage through a sieve size of at least 500 μm;
a chewing gum comprising Zn²⁺ in an amount of 0.05 to 4.0 wt %, such as 0.1 to 3.9 wt %, more preferably 0.2 to 3.8 wt %, such as 0.4 to 3.7 wt %, even more preferably 0.6 to 3.6 wt %, such as 0.8 to 3.4 wt %, and most preferably 1.0 to 3.3 wt %, such as 2 wt %.

### Example 5

The flavouring and mouth refreshing agent, levomentholum, was grinded so that it could pass though a 710 μm sieve. Levomentholum and the flow agent, silica colloid anhydrous Ph Eur, was sieved together through a 710 μm sieve.

The active ingredient, zinc bisglycinate (10%), and a flavouring agent in form of the artificial sweetener acesulfam potassium was, as a separate operation, sieved together through a 710 μm sieve.

8 kg zinc bisglycinate, 4.16 kg levomentholum, 1.88 kg silica colloid anhydrous and 360 g acesulfam potassium was subsequently mixed in a drum mixer (from the company Servolift).

Subsequently, 10 kg of the flavouring agent Coolmint flavour powder IFF was added to the above mixture whereafter the resulting mixture was transferred to the final mixing apparatus.

To this mixture, 375.8 kg of the carrier, Cafosa HiG PWD-02, which beforehand had been divided into fine particles by using a Pharmaceutical Quality Lump Breaker apparatus (a grinder from the company Frewitt) followed by sieving through a 500 μm sieve, was added and mixed in approx. 30 minutes with the other ingredients. Subsequently, the mixture was transferred to a compression apparatus (a rotary tabletting machine, type IMA Kilian), where compression was carried out under pressure (pre-pressure: approx. 8 kN, main pressure: approx. 47 kN). The working speed of the tabletting apparatus was set to 46,000 chewing gums per hour. Additionally a Fill-o-Matic™ device with a rotational speed of 10-12 rotations/min and a K-tron™ magnesium stearate spray system set to 500 g/hour was used/needed to prevent sticking of gum mixture to dies and punches during compression.

The resulting chewing gum (diameter 13 mm, round, concave), having a total weight of approx. 1000 mg, had a satisfactory smooth surface as well as excellent texture and organoleptic properties.

### Example 6

ODTs were produced according to the recipe set forth in Table 1 below for 38,461 tablets (diameter 13 mm, round, concave).

TABLE 1
ODT

Amount

| Ingredients | per tablet, mg | Blend, kg |
|---|---|---|
| Stevia glycoside | 0.37 | 0.014 |
| Magnesium stearate | 1.00 | 0.038 |
| Aerosil 200 Pharma | 1.00 | 0.038 |
| Levomentholum | 5.20 | 0.200 |
| Coolmint flavor powder IFF | 13.30 | 0.512 |
| Xylitol 200 DC | 17.00 | 0.653 |
| Zinc glycinate chelate10% | 68.00 | 2.615 |
| Vivapur 102 | 100.00 | 3.846 |
| Advantol 200 | 411.13 | 15.813 |
| Total | 617.00 | 23.730 |

Vivapur 102 is microcrystalline cellulose providing for a lower compression pressure being required to form the tablets, which in turn provides for shorter disintegration time in the oral cavity.

The flavouring and mouth refreshing agent, levomentholum, was grinded so that it could pass though a 710 μm sieve. Levomentholum, the flow agent, Aerosil 200 Pharma, magnesium stearate and the sweetener, Stevia glycoside, were sieved together through a 710 μm sieve.

Zinc glycinate chelate 10%, the flavouring agent Coolmint flavour powder IFF, Xylitol 200 DC, Vivapur 102 and Advantol 200 were added and mixed together in a drum mixer for 30 minutes.

Subsequently, the mixture was transferred to a compression apparatus (a rotary tabletting machine, type IMA Kilian), where compression was carried out under pressure (pre-pressure: approx. 3.1 kN, main pressure: approx. 12.8 kN). The working speed of the tabletting apparatus was set to 40,000 tablets per hour. Additionally a Fill-o-Matic™ device with a rotational speed of 10-12 rotations/min and a K-tron™ magnesium stearate spray system set to 500 g/hour was used/needed to prevent sticking to dies and punches during compression.

The room temperature was 18.5° C. and 12% RF

The resulting ODT tablet, having a total weight of approx. 617 mg, had a satisfactory smooth surface, hardness (approximately 27-28 N), as well as acceptable oral dissolution time (75 sec.), and organoleptic properties.

1. An orally disintegrating composition comprising a chelate comprising a metal ion and a biologically acceptable amino acid and having the general formula 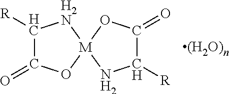
wherein M is the metal ion Zn²⁺ and R is H in the biologically acceptable amino acid glycine.

2. An orally disintegrating composition according to claim 1, wherein said chelate is controllably releasable into the oral cavity of a subject.

3. An orally disintegrating composition comprising according to claim 1, wherein the composition is based on one or more polyols, such as in the form of the commercially available composition Advantol™ 300.

4. An orally disintegrating composition according to claim 1, wherein the composition is based on one or more polyols having average particle sizes of 90-120 μm.

5. An orally disintegrating composition according to claim 3, wherein the total content of polyols in the composition is between 70 and 85 wt %.

6. An orally disintegrating composition according to claim 3, wherein the polyols are sorbitol, mannitol, xylitol, erythritol, maltitol, lactitol, isomalt and/or mixtures thereof.

7. An orally disintegrating composition according to claim 3, wherein the amounts of polyols are: 70-99.5 wt % mannitol, 0.5-30 wt % sorbitol, 0-5 wt % xylitol, 0-5 wt % erythrol, 0-30 wt % maltitol, 0-5 wt % lactitol, 0.5-20 wt % isomalt of the total amount of polyols and wherein any resulting orally disintegrating composition can comprise all possible combinations of any of said individual amounts.

8. An orally disintegrating composition according to claim 1 comprising Zn²⁺ in an amount of 0.05 to 4.0 wt %

9. An orally disintegrating composition according to claim 1, wherein all constituents of said composition are completely dissolved in the oral cavity within 180 seconds upon exposure to the saliva, preferably within 60 seconds upon exposure to the saliva.

10. An orally disintegrating composition according to claim 1, wherein the orally disintegrating composition comprises one or more saliva-inducing agent(s).

11. An orally disintegrating composition according to claim 1, wherein the orally disintegrating composition comprises one or more flavouring agent(s).

12. An orally disintegrating composition according to claim 1, wherein the orally disintegrating composition is in the form of a lozenge, a tablet, a pellet, a pill, a troche, a water-free mouth rinse ODT or similar formulations.

13. Use of an orally disintegrating composition according to claim 1 for the treatment of halitosis.

14. A chewing gum for the treatment of halitosis comprising a chelate comprising a metal ion and a biologically acceptable amino acid and having the general formula 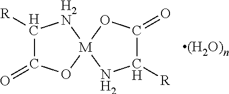
wherein K4 is the metal ion Zn²⁺ and R is H in the biologically acceptable amino acid glycine, CHARACTERISED in that the chewing gum is based on a carrier composition comprising:
(i) a gum base
(ii) sorbitol
(iii) xylitol
(iv) one or more plasticizer(s)
(v) one or more anticaking agent(s) such as the commercially available Cafosa HiG PWD-02 and/or Cafosa HiG PWD-01.

15. A chewing gum according to claim 14 wherein the ratio of the constituents of the carrier composition are:
(i) a gum base: 28-31 wt %
(ii) sorbitol: up to 100 wt %
(iii) xylitol: 8-12 wt %
(iv) one or more plasticizer(s): ≦1.5 wt %
(v) one or more anticaking agent(s): ≦2.0 wt %
based on the total weight of the carrier composition.

16. A chewing gum according to claim 14 wherein said chelate is controllably releasable into the oral cavity of a subject.

17. A chewing gum according to claim 14 comprising one or more saliva-inducing agent(s).

18. A chewing gum according to claim 14 comprising one or more flavouring agent(s).

19. A chewing gum according to claim 18, wherein the flavouring agent(s) includes an artificial sweetener such as acesulfam potassium.

20. A chewing gum according to claim 18, wherein the flavouring agent(s) includes a mouth refreshing agent such as levomentholum, coolmint flavours or similar menthol based compounds made from peppermint or other mint oils.

21. A chewing gum according to claim 14, comprising one or more flow agent(s), such as colloidal silica, and anticaking agents, such as magnesium stearate and/or talc powder or similar glidants or antiadherants.

22. A chewing gum according to claim 14, wherein the ratio between the chelate, the carrier composition and any exipients are approximately 2:94:4.

23. A chewing gum according to claim 14, wherein all exipients have particle sizes allowing passage through a sieve size of at least 710 μm.

24. A chewing gum according to claim 14, wherein all constituents of the carrier composition have particle sizes allowing passage through a sieve size of at least 500 μm.

25. A chewing gum according to claim 14, comprising Zn²⁺ in an amount of 0.05 to 4.0 wt %.
